(19) United States
(12) Patent Application Publication
(10) Pub. No.: US 2005/0043214 A1
(43) Pub. Date: Feb. 24, 2005

(54) Antiglaucomatous agent and use thereof

(76) Inventor: Martin Goerne, Hamburg (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/496,949

(22) PCT Filed: Nov. 27, 2002
(86) PCT No.: PCT/EP02/13386

(30) Foreign Application Priority Data
Nov. 27, 2001 (DE) 10158037.1

(51) Int. Cl. A61K 38/37; A61K 31/5377; A61K 31/4178

(52) U.S. Cl. 514/2; 514/235.2; 514/397; 514/651; 514/546

(57) ABSTRACT

The present invention relates to pharmaceutical compositions based on antiglaucomatous active ingredients and to the use thereof for the treatment of glaucomas. The compositions comprise lactalbumin hydrolysate or a fraction thereof. Substantially lower doses of the antiglaucomatous agents are thus possible than is conventionally necessary to achieve a particular antiglaucomatous effect.

The present invention relates to pharmaceutical compositions based on antiglaucomatous active ingredients and to the use thereof for the treatment of glaucomas.

Lactalbumin hydrolyzates have been used for some time as dietary foods. For example, a peptide mixture composed of lactalbumin, meat and soy is used in cases of maldigestion, malabsorption, in consuming processes and for strengthening. Lactalbumin hydrolyzates are distinguished by being able to be absorbed very readily and being available for metabolism in a very short time. The low molecular weight fraction in particular is particularly suitable for absorption and further metabolic utilization.

In addition, there have also been reports that degradation products said to have pharmacological effects can be obtained starting from whey protein, lactalbumin, α-lactalbumin, lactoferrin, β-lactoglobulin, lysozyme or serum albumin. Thus, DE-A 39 22 453 and PCT/EP86/00016 (WO 86/04217) for example mention an analgesic, antiglaucomatous or antimutagenic activity. It is therefore recommended that the degradation products be used for the treatment of painful inflammations of all types, neurodermatitis, arthritis, rheumatism, but also of glaucomas. The milk constituents obtainable according to DE 38 29 552 A1 are also said to have characteristic pharmacological properties and to be usable for the treatment of neurodermatitis, allergies, glaucoma and for immunostimulation. However, there is vigorous controversy about the pharmacological activity of these lactalbumin hydrolyzates. Nor has it been possible to demonstrate it in the manner necessary for clinical use.

Glaucomas are disorders of the eye whose common characteristic is that they lead to an increased intraocular pressure. A distinction is made in principle between glaucomas with obstructed filtration angle, angle-closure glaucomas, and glaucomas with open filtration angle, open-angle glaucomas. The cause of so-called inflow glaucomas is overproduction of aqueous humor, whereas so-called outflow glaucomas are based on a deterioration in the outflow of aqueous humor. Inflow glaucomas are rare in practice. Outflow glaucomas are of considerably greater practical importance.

One possible type of therapy is represented by surgical procedures whose aim is to ensure an adequate outflow of aqueous humor. However, such procedures are generally associated with an increased surgical risk, in particular a relatively high risk of blindness, also as late complication. It is intrinsic to fistulizing procedures, such as trephination, moreover that there is a continued risk of infection entering the eye via the fistula. Laser coagulation procedures such as cyclophotocoagulation, ciliary body excision and coagulation in the region of the trabecular meshwork have the disadvantage of scarring and, associated therewith, a reduction in the existing outflow.

In many cases permanent regulation of the intraocular pressure is not possible by conventional surgical procedures. Thus, the resistance to outflow of the remaining trabecular meshwork increases as glaucoma progresses, and, as a consequence, the pathological increase in intraocular pressure is resumed.

For the aforementioned reasons, not only open-angle glaucomas but also angle-closure glaucomas are treated pharmacologically to reduce the intraocular pressure. The aim of pharmacotherapy is to control the intraocular pressure as long as possible in order to delay the time of a surgical antiglaucomatous procedure or make the latter in fact possible. There are in addition angle-closure glaucomas based on anatomical changes which do not make a surgical procedure appear promising. In these cases, permanent pharmacotherapy to reduce the intraocular pressure is indicated also in the case of angle-closure glaucoma.

Known antiglaucomatous active ingredients with an intraocular pressure-reducing effect are found among the muscarine receptor agonists, the beta-receptor blockers, the carbonic anhydrase inhibitors and further active ingredients with acknowledged antiglaucomatous effect, such as guanethidine and latanoprost.

However, these intraocular pressure-reducing agents in many cases display unwanted side effects. Thus, muscarine receptor agonists may cause constriction of the pupil (miosis) and the disturbances of vision associated therewith. Beta-receptor blockers may bring about disturbances of heart rhythm and reduce tear secretion (sicca syndrome). The surface analgesic effect of beta-receptor blockers is also disadvantageous because minor injuries of the cornea cannot be perceived and contact lenses ought not be worn. Carbonic anhydrase inhibitors also have diverse unwanted side effects.

Since pharmacotherapy in most cases must be continued over a long period and often in fact life-long, active ingredient doses which are as low as possible are desirable in order to reduce side effects to a tolerable level

However, intraocular pressure-reducing agents gradually lose their main pressure-reducing effect during the treatment. It is for this reason often necessary to increase the dosage, thus increasing the unwanted side effects. For example, a chronic simple glaucoma can be treated initially by instillation into the conjunctival sac of two drops of a 0.1% timolol solution each day. This initial timolol dose must then be continually increased during the treatment, for example by administering the normally supplied more concentrated 0.25% or even 0.5% timolol solution. It is also recommended in many cases to employ a combination of various pressure-reducing substances as the intraocular pressure-reducing effect declines. However, even here, an increase in the single doses may be necessary during the further course of the treatment.

The invention is therefore based on the object of providing antiglaucomatous agents which have as few side effects as possible.

It has now been found that the effect of known antiglaucomatous active ingredients can be considerably enhanced when they are administered in combination with lactalbumin hydrolyzates.

The present invention therefore relates to pharmaceutical compositions based on at least one antiglaucomatous active ingredient, which are characterized in that they comprise lactalbumin hydrolyzate or a fraction thereof.

The compositions of the invention provide substantial therapeutic advantages. In particular, substantially lower doses of the antiglaucomatous agents are possible than is conventionally necessary to achieve a particular antiglaucomatous effect. It is usually possible to reduce the dosage of the antiglaucomatous agent while maintaining its main antiglaucomatous effect to about 2 thirds or even further. Fewer side effects attributable to the antiglaucomatous agent occur thereby. This is important especially when an antiglaucomatous treatment is indicated over a prolonged period, such as, for example, in the treatment of open-angle glaucoma. This is made possible by the enhancement of the antiglaucomatous effect of the antiglaucomatous agents by the lactalbumin hydrolyzates, and thus the amount needed to achieve a particular antiglaucomatous effect is comparatively lower.

The antiglaucomatous agents and the lactalbumin hydrolyzates can in principle be administered together in one formulation or separately in at least two different formulations. The latter possibility encompasses both simultaneous administration and administration with a time lag, i.e. taking place at different times. Simultaneous administration is preferred, especially in the form of a joint formulation.

Lactalbumins are generally known as constituent of milk and in particular of whey proteins. These are proteins which can be obtained in a manner known per se usually mixed with further milk constituents, i.e. in particular whey proteins such as lactalbumin, lactoferrin, β-lactoglobulin, lysozyme or serum albumin.

The starting material used is fresh raw milk from a domestic animal, preferably cow's milk, which can also have been subjected to a heat treatment which is now conventional in dairies, but is preferably untreated. It can have been skimmed in a conventional way, e.g. by centrifugation.

Removal of caseins can take place in a manner known per se, for example with the aid of so-called rennet or acid precipitation, in which the caseins precipitate while the whey proteins remain in solution. An alternative possibility is to remove the caseins with the aid of a membrane filtration on a microporous membrane with a pore size in the range from 0.1 to 0.6 μm, preferably 0.2 μm. The permeate (or filtrate) contains all the salts, lactose, amino acids, oligopeptides and low molecular weight peptides, while the retentate comprises virtually all the casein constituents of the milk and—unless previously skimmed—also the fat constituents thereof.

Lactalbumins can then be obtained in a manner known per se, in particular by ultrafiltration, from the milk fraction substantially freed of casein and fat. For example, the lactalbumins can be obtained as retentate on a membrane with a cut-off of from 6 to 10 000 Da. It is possible to use for example the product Lactalbumin 75 marketed by Milei.

According to a particular aspect, preference is given according to the invention to the use of lactalbumin hydrolyzates or fractions thereof whose weight average molecular weight is in a range from about 50 to 1200, preferably from about 60 to 800 and in particular from about 80 to 500. Particularly preferred lactalbumin hydrolyzates have a molecular weight distribution with an upper molecular weight limit of about 760 and in particular of 500.

The lactalbumin hydrolyzate to be used according to the invention can be obtained by enzymatic hydrolysis of lactalbumin or a lactalbumin-containing mixture (lactalbumin preparation). For this purpose, an aqueous lactalbumin suspension is treated with at least one protease and, if desired, a lipase.

The hydrolysis generally takes place completely, i.e. until the enzymatic reaction is complete. Depending on the enzyme or enzyme mixture, a degree of hydrolysis of about 20% to about 90%, preferably of about 40% to about 80% and in particular about 75%, can be achieved.

The degree of hydrolysis can be determined and calculated in a manner known per se. It is thus possible to follow the progress of the hydrolysis during the reaction. The formol titration method has proved particularly suitable, according to which the number of free amino groups can be estimated by titration with sodium hydroxide.

The conditions which can be chosen for the purpose of hydrolysis, such as the enzymes employed and the activity thereof, the temperature, the pH, the amount of lactalbumin and the respective concentrations of the reaction mixture, are mutually dependent and can be optimized in the manner of a skilled worker.

The protease(s) used are preferably papain, pancreatin, chymotrypsin and/or trypsin. Optionally, it is also possible to employ proteases obtained from fungi and/or bacteria, especially in addition to the aforementioned proteases. The fungal protease is selected in particular from the proteases from Tritirachium species, in particular Tritirachium alba, proteases from Aspergillus species, in particular Aspergillus saitoi, Aspergillus sojae, Aspergillus oryzae and/or from Rhizopus species, in particular Newlase, and the bacterial protease is selected in particular from proteases from Streptomyces species, in particular Streptomyces caespitosus, Streptomyces griseus (Pronase E.), from Bacillus subtilis types, in particular Subtilopeptidase A (Carlsberg subtilisin), and from Bacillus polymyxa. Further proteases which can be used are proteases obtainable from pineapple, such as bromelain.

If the lactalbumin preparation also contains noticeable quantities of starch and starch-like products, it is advantageous additionally to use a suitable amylase, preferably an α-amylase, e.g. from a subtilis type. The activity optimum of such an amylase is usually at a pH of from 5.7 to 7.2. The reaction temperature can be up to 75° C.

Additional use of a lipase is usually also necessary only if the lactalbumin preparation contains noticeable quantities of fats.

The enzymes are advantageously used in an amount of about 0.01 to 2% by weight based on the suspension.

The protease preferably used is papain or a mixture of approximately equal parts by weight of pancreatin and papain.

A further advantageous protease mixture is a mixture of approximately equal proportions of papain, pancreatin and a bacterial or fungal protease, e.g. the bacterial protease Pronase E from Streptomyces griseus or the fungal protease product “Newlase” from an Aspergillus species. Likewise advantageous is a protease mixture composed of approximately equal proportions of papain, pancreatin and bromelain.

It is particularly advantageous to use, based on approximately 50 kg of lactalbumin as dry matter, approximately 100 to 1000 g, preferably approximately 300 to 700 g and in particular approximately 500 g of papain; approximately 100 to 1000 g, preferably approximately 300 to 700 g and in particular approximately 500 g of pancreatin; and, optionally, approximately 100 to 1000 g, preferably approximately 300 to 700 g, in particular approximately 500 g of a bacterial or fungal protease, in particular Newlase, or of a protease obtainable from pineapple, in particular bromelain.

An approximately 1 to 10% by weight and advantageously a 2 to 5% by weight suspension of lactalbumin in water is expediently used as starting material.

Reaction times in the region of hours are usual, in particular approximately 1 to 8 hours and advantageously approximately 2 to 4 hours; elevated reaction temperatures, approximately 30 to 70° C. and advantageously in the region of the temperature optimum of the enzymes employed; pH values in the slightly acidic to slightly alkaline range, in particular approximately at 6.6 to 7.8 and preferably in the pH optimum of the enzymes employed. On use of enzyme mixtures and especially those with different temperature or pH optima, the respective enzymes can be allowed to act on the lactalbumin at different times by adding part of the enzyme mixture to the reaction mixture at a later time, and adapting the conditions appropriately. In particular, the more thermally stable enzymes can be added after initial, at least partial hydrolysis at moderate temperature employing the less thermally stable enzymes, and subsequently the hydrolysis can be continued using the more thermally stable enzymes at higher temperature. Thus, in the present case, papain and/or pancreatin can be allowed to act on the lactalbumin initially at temperatures in the range from 35 to 38° C. and preferably at approximately 37° C., and subsequently the hydrolysis can be continued through the action of a suitable bacterial and/or fungal protease or of a protease obtainable from pineapple at temperatures in the range of approximately 50 to 75° C. and preferably at approximately 60° C. The procedure for this purpose is expediently such that the protease is added to the reaction mixture and then the temperature is gradually raised. It may be desired in particular for the temperature to be raised gradually to the final temperature during the reaction time which still remains.

Thus, in a particular embodiment of the present invention, there is use of a lactalbumin hydrolyzate which is obtainable by allowing an enzyme mixture containing papain and pancreatin to act on lactalbumin in aqueous suspension at approximately 35 to 38° C. for from 1 to 3 hours; adding a bacterial or fungal protease or a protease obtainable from pineapple, and raising the temperature within from 1 to 4 hours to approximately 60° C., and obtaining the hydrolyzate in a manner known per se.

The procedure for obtaining the hydrolyzate is expediently such that initially the enzymes are inactivated. The reaction mixture can be briefly heated for this purpose. The usual temperatures are in the range from 80 to 85° C., in which case inactivation is achieved after only a few minutes. An alternative possibility is also to inactivate the enzymes by sterilization at ultrahigh temperatures. A few seconds at approximately 130° C. are generally sufficient.

As a consequence, the enzymes usually precipitate and the resulting precipitate is preferably removed after cooling the reaction mixture. For this, it is possible for the reaction mixture to be filtered directly or initially concentrated, for example in vacuo or by spray drying, and for the residue then to be taken up in a suitable solvent and the resulting suspension to be filtered. Solvents suitable in this connection are lower alcohols such as methanol, isopropanol and preferably ethanol or mixtures thereof with water and also organic solvents, especially chloroform.

In this way, for example, approximately 200 mg of lactalbumin hydrolyzate in ethanolic solution are obtained from 5 g of lactalbumin, this hydrolyzate frequently resulting as mixture of the actual lactalbumin peptides as main constituent and further additional constituents. The nature and quantity of the additional constituents, especially fats, depend in particular on the starting material used for the hydrolysis.

In a particular embodiment of the present invention, certain fractions of lactalbumin hydrolyzates which are obtainable in particular from those described above by extraction are used.

It is particularly preferred for the lactalbumin hydrolyzates to be initially defatted before the extraction. Suitable for this purpose are, in a known manner, solvents such as, for example, petroleum ether, which can expediently be employed in a continuous, exhaustive extraction to remove the fat constituents.

Suitable extractants are predominantly polar solvents such as lower alcohols, especially methanol, ethanol, isopropanol and mixtures thereof with water.

Preferred fractions can be obtained by extracting lactalbumin hydrolyzate with absolute ethanol, filtering and obtaining the filtrate. The ethanolic solution contains a lactalbumin hydrolyzate fraction A (ethanol extract) which can be obtained as solid by concentration. This extraction of the preferably defatted lactalbumin hydrolyzate with ethanol preferably takes place by continuous, exhaustive extraction. This also includes removal of nonextractable, i.e. ethanol-insoluble, constituents to obtain an extract solution which can subsequently be concentrated to dryness in order to obtain the lactalbumin hydrolyzate fraction which can be extracted in this way as solid.

Particularly preferred fractions can be obtained by extracting lactalbumin hydrolyzate or fractions thereof with isopropanol, filtering and obtaining the filtrate. The isopropanolic solution contains a lactalbumin hydrolyzate fraction B (isopropanol extract) which can be obtained as solid by concentration. A preferred isopropanol extract can be obtained by employing the residue A obtained by ethanol extraction, and extracting appropriately with isopropanol (lactalbumin hydrolyzate fraction B1).

For this purpose, lactalbumin hydrolyzate and especially the fraction A obtained by the previously described ethanol extraction can be taken up in isopropanol to result in an easily stirrable mixture. For example, the lactalbumin hydrolyzate or a fraction thereof can be stirred with approximately 10 times the volume of isopropanol. The result is usually a suspension which is filtered to remove solids. It has proved to be expedient initially to store the mixture preferably in the cold for some hours up to a few days, and then to filter off the resulting solid phase. About 2 days at a temperature of from 2° C. to 4° C. leads to good results. The filtrate is subsequently concentrated to dryness, it being possible to proceed in accordance with the above statements about the concentration of the ethanol extract. It has additionally proved to be expedient to remove isopropanol residues by repeatedly taking up the residue in ethanol and freeing from solvent by concentration. For example, good results are achieved by taking up in sufficient ethanol and subsequently concentrating three times.

Particularly preferred fractions can be obtained by residue A obtained by ethanol extraction being taken up in absolute ethanol and adjusted with water to an ethanol content of approximately 20 to 60% by volume, preferably of approximtely 30 to 50% by volume and in particular of approximately 40% by volume. The result is usually a suspension, whose solid phase can be removed by filtration. For this, it is expedient for the mixture which is, optionally, initially still in the form of a solution to be stored, preferably in the cold, for several hours to a few days, and then to be filtered. In this case too, 2 days at 2° C. to 4° C. leads to good results. The hydroethanolic solution contains a lactalbumin hydrolyzate fraction C which can be obtained as solid by concentration. Preferred lactalbumin hydrolyzate fractions can be obtained by employing the residue B, and in particular B1, obtained by isopropanol extraction, and proceeding correspondingly, with a lactalbumin hydrolyzate fraction C1 or, in particular, a lactalbumin hydrolyzate fraction C2 being obtained.

The concentration to dryness usually takes place in a manner known per se, e.g. in vacuo or by spray drying. The concentration can expediently take place by evaporation of the ethanol at slightly elevated temperature, for example at approximately 40° C. If desired, the residue obtained is dried further, for example in vacuo. Other, similarly mild methods are likewise suitable.

Similar statements apply to the removal of the isopropanol and hydroalcoholic mixtures.

The lactalbumin hydrolyzate fractions are characterized by an increased content of lactalbumin hydrolyzate peptides, a fraction of the initially employed lactalbumin hydrolyzate. According to a particular aspect, this peptide fraction includes 2 peptides (peptide A and peptide B), the proportion of each of which in the peptide fraction is larger than the proportion of any other peptide in this fraction. The two peptides A and B are, in particular, tri- and/or tetrapeptides. According to a further aspect, peptides A and B are characterized by a chromatographic behavior allowing them to be purified by reverse phase chromatography, e.g. on use of an RP 18 column (5μ material) by elution with an acetonitrile/water/trifluoroacetic acid gradient, with sufficiently different retention times.

Accordingly, peptides A and B can be enriched and obtained from lactalbumin hydrolyzates or fractions thereof.

Particular lactalbumin hydrolyzate peptides of lactalbumin hydrolyzates of the invention are, according to a further aspect, characterized by a molecular ion peak (m+H⁺) in mass spectrometric TOF analysis of m/z=247, 269, 341 and/or 399. The peptides with the molecular ion peaks of 341 and 399 are particularly important. Particular lactalbumin hydrolyzate peptides are therefore to be selected in particular from tripeptides having two proline residues and glutamine or lysine as further amino acid residue and tetrapeptides having a leucine residue or an isoleucine residue, and a proline residue, an alanine residue and a valine residue.

According to one aspect, predominant proportions of lactalbumin hydrolyzates or fractions thereof to be used according to the invention are freely soluble both in chloroform and in water. These proportions are preferably at least 90% by weight, preferably at least 95% by weight and in particular at least 98% by weight, of the respective lactalbumin hydrolyzate or fraction thereof.

Antiglaucomatous agents in the widest sense include active ingredients which act to reduce the intraocular pressure. These include certain muscarine receptor agonists, beta-receptor blockers, carbonic anhydrase inhibitors and further active ingredients known to have an antiglaucomatous effect.

Antiglaucomatous active ingredients which can be particularly used according to the invention are selected from:

a) muscarine receptor agonists such as acetylcholine chloride, carbachol and bethanicol, and the corresponding trimethylammonium derivatives with further pharmaceutically suitable counter ions, pilocarpine, aceclidinum, eserine, neostigmine, galantamine, brimonidine, clonidine, dipivefrine and apraclonidine, and derivatives thereof, optionally in salt form, e.g. pilocarpine nitrate, pilocarpine hydrochloride, neostigmine bromide, brimonidine tartrate, clonidine hydrochloride and dipivefrine hydrochloride;

b) beta-receptor blockers such as carteolol, timolol, metipranolol, betaxolol, befunolol, pindolol, levobunolol and bupranolol, and derivatives thereof are, optionally in salt form, e.g. carteolol hydrochloride, timolol maleate, timolol hydrogen maleate, metipranolol hydrochloride, betaxolol hydrochloride, befunolol hydrochloride, and levobunolol hydrochloride;

c) carbonic anhydrase inhibitors such as brinzolamide, dorzolamide, acetazolamide,-methazolamide, ethoxzolamide and diclofenamide and derivatives thereof, optionally in salt form;

d) further substances acknowledged to have antiglaucomatous effect, such as demecarium bromide, diethyl p-nitrophenyl phosphate, diisopropyl fluorophosphate and ecothiopate iodide, and derivatives thereof, optionally in salt form; guanethidine and derivatives thereof, optionally in salt form.

The following antiglaucomatous agents are preferred according to the invention:

timolol and derivatives thereof, optionally also in salt form.

An effective amount of lactalbumin hydrolyzate or of a fraction thereof and an effective amount of at least one antiglaucomatous agent, usually formulated in accordance with pharmaceutical or veterinary practice, is administered according to the invention to the individual to be treated, preferably a mammal, especially a human and also an agricultural animal or pet.

Ordinarily, a suitable dose is administered each day, one or more times, optionally together or alternately with other active ingredients or active ingredient-containing products, so that the daily dose administered to an individual to be treated is approximately 2 mg to 100 g, preferably approximately 10 mg to 80 g, advantageously approximately 20 mg to 60 g, and in particular approximately 50 mg to 60 g, of lactalbumin hydrolyzate on oral administration, approximately 0.5 mg to 80 g, preferably approximately 8 mg to 70 g, advantageously approximately 20 mg to 70 g, and in particular approximately 30 mg to 40 g, of lactalbumin hydrolyzate on parenteral administration and approximately 0.5 mg to 100 g, preferably approximately 1 mg to 80 g, advantageously approximately 5 mg to 60 g, and in particular approximately 10 mg to 60 g, of lactalbumin hydrolyzate on topical use. The amounts of lactalbumin fractions to be administered are usually below the aforementioned dosages. Thus, the dosage for the lactalbumin hydrolyzate constituents may be chosen to be approximately a factor of 5 to 20 and the dosage for the lactalbumin hydrolyzate peptides may be chosen to be approximately a factor of 10 to 30 lower.

It is moreover possible to choose the dosage of the antiglaucomatous agent according to the invention to be lower than conventionally recommended for achieving the main antiglaucomatous effect (pressure reduction). Accordingly, the daily dose recommended in particular for the treatment of glaucomas can be reduced by at least a factor of 1.5 and advantageously by at least a factor of 2. If, for example, it is already necessary to administer 2 drops of a 0.5% strength timolol solution each day to a patient with chronic open-angle glaucoma in order to reduce the intraocular pressure to below 21 mmHg, it is possible according to the invention to achieve the same therapeutic effect with a combination of 0.2 mg of lactalbumin hydrolyzate fraction C1 or C2 and 2 drops of an only 0.1% strength timolol solution.

The extent to which the dose of an active ingredient acknowledged to have an antiglaucomatous effect can be reduced when it is used according to the invention in combination with lactalbumin hydrolyzate or a fraction thereof can be demonstrated in particular by controlled, randomized double-blind studies. It is possible to use for this purpose for example clinical variables which document in accordance with scientific knowledge the progress of the therapy of the pathological state investigated. Animal models can also be used in addition.

For example, on use according to the invention of a combination of timolol and a lactalbumin hydrolyzate fraction on the one hand and of a 5-times higher timolol dose without use of a lactalbumin hydrolyzate fraction of the invention for the treatment of chronic simple glaucoma, the therapy is assessed by documentation of the intraocular pressure, the visual field and the photographic monitoring of the optic disk. Monitoring of the visual field and optic disk serve to exclude possible occult increases in intraocular pressure which may occur outside the time of measurement of the intraocular pressure. The degree of reduction in the dose is ascertained by referring to reference values for the therapy of chronic simple glaucoma.

The amounts and proportions of active ingredients are based on the active active ingredient, so that an appropriate recalculation is necessary for salts and derivatives.

The compositions of the invention are particularly suitable for the treatment of glaucoma. The present invention therefore also relates to the use of lactalbumin hydrolyzates or fractions thereof in combination with at least one antiglaucomatous agent for the treatment of glaucoma.

The glaucoma treatment of the invention aims in particular to reduce the intraocular pressure. It is therefore a symptomatic type of treatment. The intraocular pressure in the healthy eye, which is produced by a balance between continuous production and outflow of aqueous humor, is in the region of about 9 mmHg up to about 21 mmHg, in each case measured by applanation.

A glaucoma to be treated according to the invention is characterized in particular by an intraocular pressure, measured by applanation, of more than 21 mmHg. This particularly applies when such elevated intraocular pressures are measured repeatedly, and thus the intraocular pressure is chronically elevated. However, the treatment of the invention also includes low-pressure glaucomas in which functional deficits of the eye caused by glaucoma occur even when intraocular pressures are in the upper part of the normal range, i.e. 20 mmHg measured by applanation. Indications of incipient functional deficits of the eye caused by glaucoma, such as damage to the optic disk, especially optic nerve atrophy and/or excavation of optic disk, losses of visual field, diminution in visual acuity and a greenish reflection from the lens, support the diagnosis of glaucoma and serve to differentiate glaucoma from ocular hypertension, which essentially does not lead to pathological changes, i.e. in particular not to the functional deficits of the eye caused by glaucoma and described above, when the intraocular pressures are moderately elevated, in particular measured by applanation, up to about 26 mmHg.

The glaucomas which can be treated according to the invention include in particular glaucomas with open filtration angle (open-angle glaucomas, also referred to as wide-angle glaucomas). These include in particular chronic simple glaucoma. The latter is characterized in particular by an elevated content of hyaluronic acid in the trabecular meshwork. Also present in the trabecular meshwork are globulins and plasma cells. With increasing age there may be a thickening of the trabecular partitions and/or a displacement of the intertrabecular lamina.

Further open-angle glaucomas which can be treated according to the invention are pigmentary glaucoma (in this case the pigment epithelium of the iris increasingly loses pigment which accumulates, like tealeaves, in an outflow in the trabecular meshwork and increasingly blocks the latter); pseudoexfoliation glaucoma, also referred to as capsular glaucoma (in this case there is the formation of pseudoexfoliative material which, in a similar way to pigmentary glaucoma, accumulates in the trabecular meshwork and increases the outflow resistance); late juvenile glaucoma; forms of infantile glaucoma (buphthalmos); especially Sautter's juvenile glaucoma; glaucoma associated with high myopia; glaucoma associated with-diabetes mellitus; and cortisone-inducible glaucoma.

The open-angle glaucomas which can be treated according to the invention also include secondary glaucomas which have developed as a result of another disorder, for example glaucomas resulting from inflammatory changes in the eye; glaucomas resulting from formation of new vessels (neovascular glaucoma); glaucomas resulting from uveitis, from iridocyclitis, from Fuchs' heterochromic iridocyclitis, from a Kraupa-Posner-Schlossmann syndrome and from a keratouveitis, for example associated with herpes simplex infections or ophthalmic zoster; in addition glaucomas resulting from a Kirsch effect due to alpha-chymotrypsin; glaucomas resulting from blood in the anterior chamber; post-contusion glaucomas resulting from a contusional deformity of the filtration angle; macrophage glaucomas; glaucomas associated with intraocular tumors; glaucomas associated with epithelial ingrowth into the filtration angle; glaucomas associated with orbital processes resulting from elevated episcleral venous pressure, for example in the case of myositis or of arteriovenous fistula.

Besides open-angle glaucomas, it is also possible according to the invention to treat angle-closure glaucomas. It is thus possible to postpone the time of a surgical antiglaucomatous procedure or in fact make it possible (preoperative reduction in the intraocular pressure). In particular, the pharmaceutical treatment of the invention may be indicated for certain types of angle-closure glaucoma if a surgical procedure does not appear promising.

A particular aspect of a treatment in the sense according to the invention relates to the treatment of chronic disorders, i.e. in particular of chronic open-angle glaucoma (wide-angle glaucoma).

The invention also relates to the production of compositions for the treatment of an individual, preferably of a mammal, in particular of a human and of an agricultural animal or pet.

Compositions of the invention are usually based on an active ingredient combination and, optionally, a formulation base.

The formulation base of formulations of the invention comprises physiologically acceptable excipients. Physiologically acceptable excipients are those known to be usable in the pharmacy sector, in food technology and adjacent areas, in particular those listed in relevant pharmacopeas (e.g. DAB, Ph. Eur., BP, NF), and also other excipients whose properties do not preclude physiological use.

Suitable excipients may be: wetting agents; emulsifying and suspending agents; preservatives; antioxidants; antiirritants; chelating agents; tablet-coating aids; emulsion stabilizers; film formers; gel formers; masking odors; masking flavors; resins; hydrocolloids; solvents; solubilizers; neutralizers; permeation enhancers; pigments; quaternary ammonium compounds; refatting and superfatting agents; ointment, cream or oil bases; silicone derivatives; spreading aids; stabilizers; sterilants; suppository bases; tablet excipients such as binders, fillers, lubricants, disintegrants or coatings; propellants; desiccants; opacifiers; thickeners; waxes; plasticizers; white oils. An arrangement concerning this is based on expert knowledge as set forth for example in Fiedler, H. P., Lexikon der Hilfsstoffe für Pharmazie, Kosmetik und angrenzende Gebiete, 4th edition, Aulendorf: ECV-Editio-Cantor-Verlag, 1996.

Examples of suitable pharmaceutical formulations are solid pharmaceutical forms such as oral powders, dusting powders, granules, tablets, especially film-coated tablets, pastilles, sachets, cachets, sugar-coated tablets, capsules such as hard and soft gelatin capsules, suppositories or vaginal pharmaceutical forms, semisolid pharmaceutical forms such as ointments, creams, hydrogels, pastes or patches, and liquid pharmaceutical forms such as solutions, emulsions, especially oil-in-water emulsions, suspensions, for example lotions, preparations for injection and infusion, eye drops. Implanted delivery devices can also be used to administer active ingredients of the invention. A further possibility is also to use liposomes or microspheres. Preference is given to liquid and semisolid pharmaceutical forms for topical and, in particular, intraocular administration, especially solutions as drops (eyedrops) and gels or ointments for instillation or massaging into the conjunctival sac, in addition solid pharmaceutical forms.

The formulations may be administered for example by the oral, rectal, topical, in particular transdermal, parenteral, in particular subcutaneous, intravenous, intramuscular, intraocular or intranasal route. Preference is given to topical, especially intraocular, and oral and, if necessary, also intravenous administration.

The formulations are produced usually by mixing or diluting the active ingredients with a suitable excipient. Excipients may be solid, semisolid or liquid materials which serve as vehicle, carrier or medium for the active ingredient. Admixture of further excipients, if necessary, takes place in a manner known per se. It is possible to carry out shaping steps, optionally in conjunction with mixing processes, e.g. a granulation, compression and the like.

It is possible in particular for the active ingredient components to be formulated together. However, they can also be initially processed separately and then be combined in a compartmented, e.g. multilayer, pharmaceutical form. It is possible in this way to take account of possible active ingredient incompatibilities and different active ingredient properties such as bioavailability, stability, solubility and the like.

Besides combination products, the invention also relates to corresponding single-drug products in the form of commercial packs from which the combined use of the invention is to be taken.

The present invention is explained in detail by means of the following examples, without being restricted thereto.

### Preparation of Lactalbumin Hydrolyzate and Fractions thereof

### EXAMPLE 1

### Preparation of a Lactalbumin Hydrolyzate

5 g of lactalbumin are suspended in 130 ml of water, 50 mg of papain and 50 mg of pancreatin are added, the mixture is heated to 35-37° C. and stirred at this temperature for about 2 hours, 50 mg of a commercially available fungal protease, e.g. Newlase, a Rhizopus protease with an activity of approximately 0.5 U/mg of mass, are added, and the temperature is slowly (over the course of 2-3 hours) raised to 60° C. The temperature is then increased briefly to 80° C., the cloudy solution is allowed to cool and is concentrated in vacuo, and the residue is taken up in ethanol, filtered and concentrated in vacuo. About 0.2 g of lactalbumin hydrolyzate is obtained.

### EXAMPLE 2

### Defatting of the Lactalbumin Hydrolyzate

100 g of the lactalbumin hydrolyzate obtained in example 1 are defatted in a continuous, exhaustive reaction with 2 1 of petroleum ether at a temperature of approximately 22° C.

### EXAMPLE 3

### Obtaining a Lactalbumin Hydrolyzate Fraction A

5 g of the defatted lactalbumin hydrolyzate obtained in example 2 are extracted with 100 ml of absolute ethanol at a temperature of 22° C. in a continuous, exhaustive reaction. The ethanol extract is evaporated at 40° C. and dried in vacuo.

### EXAMPLE 4

### Obtaining a Lactalbumin Hydrolyzate Fraction B

500 g of the lactalbumin hydrolyzate fraction A obtained in example 3 are stirred with 10 times the volume of isopropanol and stored at a temperature of 2° C. to 4° C. for 2 days. The clear solution from filtration or centrifugation is concentrated to dryness in vacuo at 50° C.

Isopropanol residues are removed by taking up the residue in 2 1 of ethanol and evaporating the ethanol at 40° C. in vacuo. This procedure is repeated 2-3 times. About 400 g of a lactalbumin hydrolyzate fraction B1 are obtained as a yellowish powder.

### EXAMPLE 5

### Obtaining a Lactalbumin Hydrolyzate Fraction C

The lactalbumin hydrolyzate fraction B1 freed of isopropanol residues in example 4 is dissolved in 2 1 of absolute ethanol, and the ethanol content is adjusted to 40% by volume by adding water. The solution is then stored at 2-4° C. for 2 days. The clear solution resulting from filtration or centrifugation is concentrated to dryness at 40° C. in vacuo. About 300 g of a lactalbumin hydrolyzate fraction C2 are obtained as a yellowish powder.

Mass spectrometic analysis of the lactalbumin hydrolyzate fraction C2 reveals the following peaks (m/z+/−0.5; TOF MS ES+):

72.09; 86.10; 98.99; 120.09; 127.08; 149.03; 185.18; 188.08; 199.19; 229.17; 244.18; 247.12; 269.17; 286.99; 288.98; 341.23; 349.01; 360.21; 399.27; 431.25; 453.23; 469.23; 496.35; 525.36; 527.37; 557.35; 565.28; 569.30; 587.26; 587.28; 609.38; 649.35; 654.41; 682.42; 683.36; 723.37; 724.36; 763.40; 764.43; 772.42; 773.44; 797.42; 837.47; 838.49; 860.45; 871.52; 872.51; 889.54; 926.55; 927.51; 948.52; 949.46; 951.53.

Investigation of the lactalbumin hydrolyzate fraction C2 by HPLC analysis (reverse phase RP-18; 5μ material; elution with an acetonitrile/water/trifluoroacetic acid gradient; detection at 205 nm) shows besides free amino acids a peptide mixture which, in the oligopeptide region (dipeptides to decapeptides), comprises two main fractions which, relative to the hydrophobic amino acids which elute at about 7.5 min, especially tryptophan, elute at about 16 min and about 19.5 min, respectively. These main fractions are, according to their chromatographic behavior, tri- or tetrapeptides, so that it is to be assumed that a substantial constituent of the lactalbumin hydrolyzates of the invention is formed by two peptides, probably tri- and/or tetrapeptides.

These peptides can be isolated by an appropriate preparative HPLC.

### Pharmaceutical Compositions

### EXAMPLE 6

a) Timolol eyedrops:

| | | |
|---|---|---|
| Timolol hydrogen maleate | 1.37 | mg |
| Lactalbumin hydrolyzate fraction from Ex. 5 | 5 | mg |
| Benzalkonium chloride | 0.05 | mg |
| Sodium dihydrogen phosphate | | |
| Sodium monohydrogen phosphate | | |
| Water | ad 1 | g |

To treat chronic simple glaucoma, 1 to 2 drops are to be taken in the conjunctival sac each day.

### EXAMPLE 7

Patients with glaucoma which had led to glaucomatous damage were treated. All the patients had previously been treated with timolol 0.5% eyedrops, and in some cases with a combination therapy of timolol 0.5% and another antiglaucomatous agent. The initial state of the intraocular pressure was measured during the previous therapy by determining the average of the last six measurements during the previous therapy.

Two groups of patients were then treated according to the invention. Group 1 included patients who did not have normal intraocular pressures during maximum drop therapy, i.e. a combination therapy with timolol 0.5% and another antiglaucomatous agent (e.g. pilocarpine 2%, clonidine ¼%, carbachol 2%). They formed the group of decompensated glaucomas.

Group 2 comprised patients who had normalized intraocular pressures on treatment with timolol 0.5%. They formed the group of compensated glaucomas.

The patients from group 1 and 2 were then treated with eyedrops which contained 0.1% timolol and 0.5% lactalbumin hydrolyzate fraction from example 5 twice a day by instilling one drop of the solution into the conjunctival sac on each occasion.

A third group formed of 11 patients was treated in the same manner with eyedrops which contained only 0.5% lactalbumin hydrolyzate fraction from example 5.

The intraocular pressure was checked initially after one day, after two days and then weekly. The measurements of intraocular pressure took place at various times of day (morning, midday, late afternoon) by applanation tonometry.

In total, 18 patients with an average age of 56.6 years, of whom 8 were male and 10 patients were female, were treated for 9 weeks.

The diagnosis for the patients before the treatment according to the invention was as follows:

| simple gl. | contusion gl. | neovasc. gl | to i.o. infl. |
|---|---|---|---|
| 9 | 2 | 5 | 2 |

Group 1: decompensated >21
mmHg appl.

| | | | |
|---|---|---|---|
| 6 | 2 | 5 | 1 |

Group 2: compensated

| | | | |
|---|---|---|---|
| 3 | 0 | 0 | 1 |

Number of treated eyes

| | | | |
|---|---|---|---|
| 18 | 2 | 7 | 2 |

Group 1: decompensated >21
mmHg appl.

| | | | |
|---|---|---|---|
| 12 | 2 | 7 | 1 |

Group 2: compensated

| | | | |
|---|---|---|---|
| 6 | 0 | 0 | 1 |

The following average pressures were measured before the treatment:

(I) Pressure in the group of decompensated glaucomas on treatment with timolol 0.5% or combination therapy: 26.4 mmHg by applanation (range of variation 3.6 mmHg)

(II) Pressure for the compensated glaucomas on treatment with timolol 0.5%: 19.2 mmHg by applanation (range of variation 1.6 mmHg)

Results after treatment according to the invention:

In 11 cases in group 1 it was possible to reduce the applanation pressure below 21 mmHg by the treatment according to the invention. This was achieved in all 4 cases in group 2. Control was not possible only for 2 patients with chron. simple Gl. and 1 patient with neovascular glaucoma in group 1. No significant reduction in pressure was found in group 3.

It was thus possible to control the pressure with the combination of the invention in 15 of 18 cases.

In all these 15 cases there was superiority over the previous treatment with timolol 0.5% or a previous combination therapy with timolol 0.5% and another pressure-reducing agent.

The 14 glaucomas which had become resistant to timolol 0.5% (group 1) are of particular interest. For these glaucomas, the combination of the invention is superior to 5 times the timolol dose and superior to a combination therapy with other antiglaucomatous agents including timolol 0.5%.

1-10. (canceled).

11. A pharmaceutical composition comprising at least one antiglaucomatous active ingredient and lactalbumin hydrolysate or a fraction thereof, wherein the antiglaucomatous active ingredient is selected from the group consisting of acetylcholine chloride, carbachol, bethanicol, pilocarpine, aceclidinum, eserine, neostigmine, galantamine, brimonidine, clonidine, dipivefrine, apraclonidine, carteolol, timolol, metipranolol, betaxolol, befunolol, pindolol, levobunolol, bupranolol, brinzolamide, dorzolamide, acetazolamide, methazolamide, ethoxzolamide, diclofenamide, demecarium bromide, diethyl p-nitrophenyl phosphate, diisopropyl fluorophosphate, ecothiopate iodide and derivatives thereof, optionally also in salt form.

12. The pharmaceutical composition as claimed in claim 11, wherein the lactalbumin hydrolysate or the fraction thereof has a weight average molecular weight of approximately 50 to 1200.

13. The pharmaceutical composition as claimed in claim 11, wherein the lactalbumin hydrolysate is obtainable by enzymatic hydrolysis with papain, pancreatin and at least one bacterial protease.

14. The pharmaceutical composition as claimed in claim 11, wherein the lactalbumin hydrolysate fraction is obtainable by extraction of a lactalbumin hydrolysate with ethanol and obtaining the ethanol extract, if desired as dry extract.

15. The pharmaceutical composition as claimed in claim 11, wherein the lactalbumin hydrolysate fraction is obtainable by extraction of a lactalbumin hydrolysate with ethanol, concentration of the extract to dryness, extraction of the resulting ethanol dry extract with isopropanol and obtaining the isopropanol extract, if desired as dry extract.

16. The pharmaceutical composition as claimed in claim 11, wherein the lactalbumin hydrolysate fraction is obtainable by extraction of a lactalbumin hydrolysate with ethanol, concentration of the extract to dryness, extraction of the resulting ethanol dry extract with isopropanol, concentration of the extract to dryness, taking up the resulting isopropanol dry extract in ethanol, addition of water until the ethanol:water ratio is approximately 20:80 to 60:40, removal of the precipitate and obtaining the hydroethanolic extract, if desired as dry extract.

17. The pharmaceutical composition as claimed in claim 11, wherein the antiglaucomatous active ingredient is selected from timolol and derivatives thereof, optionally also in salt form.

18. A method for the treatment of glaucomas, where at least one antiglaucomatous active ingredient is administered in combination with lactalbumin hydrolysate or a fraction thereof to a patient, wherein the antiglaucomatous active ingredient is selected from the group consisting of acetylcholine chloride, carbachol, bethanicol, pilocarpine, aceclidinum, eserine, neostigmine, galantamine, brimonidine, clonidine, dipivefrine, apraclonidine, carteolol, timolol, metipranolol, betaxolol, befunolol, pindolol, levobunolol, bupranolol, brinzolamide, dorzolamide, acetazolamide, methazolamide, ethoxzolamide, diclofenamide, demecarium bromide, diethyl p-nitrophenyl phosphate, diisopropyl fluorophosphate, ecothiopate iodide and derivatives thereof, optionally also in salt form.

19. The method of claim 18, wherein the antiglaucomatous active ingredient and the lactalbumin hydrolysate or the fraction thereof are administered simultaneously or with a time lag, together in one formulation or separately in at least two different formulations.

20. The pharmaceutical composition as claimed in claim 12, wherein the lactalbumin hydrolysate or fraction thereof has a weight average molecular weight of approximately 60 to 800.

21. The pharmaceutical composition as claimed in claim 12, wherein the lactalbumin hydrolysate or fraction thereof has a weight average molecular weight of approximately 80 to 500.

22. The pharmaceutical composition as claimed in claim 11, wherein the lactalbumin hydrolysate fraction is obtainable by extraction of a lactalbumin hydrolysate with ethanol, concentration of the extract to dryness, extraction of the resulting ethanol dry extract with isopropanol, concentration of the extract to dryness, taking up the resulting isopropanol dry extract in ethanol, addition of water until the ethanol:water ratio is approximately 30:70 to 50:50, removal of the precipitate and obtaining the hydroethanolic extract, if desired as dry extract.

23. The pharmaceutical composition as claimed in claim 11, wherein the lactalbumin hydrolysate fraction is obtainable by extraction of a lactalbumin hydrolysate with ethanol, concentration of the extract to dryness, extraction of the resulting ethanol dry extract with isopropanol, concentration of the extract to dryness, taking up the resulting isopropanol dry extract in ethanol, addition of water until the ethanol:water ratio is approximately 40:60, removal of the precipitate and obtaining the hydroethanolic extract, if desired as dry extract.
